(19) United States
(12) Patent Application Publication
(10) Pub. No.: US 2003/0215872 A1
(43) Pub. Date: Nov. 20, 2003

(54) Screening apparatus and method for making

(75) Inventor: William G. Clark, Pittsford, NY (US)

Correspondence Address: Stephen B. Salai, Esq., Harter, Secrest & Emery LLP, 1600 Bausch & Lomb Place, Rochester, NY, 14604-2711, US

(73) Assignee: Clark-MXR, Inc., Dexter, MI

(21) Appl. No.: 10/151,612

(22) Filed: May 20, 2002

(51) Int. Cl. C12Q 1/00; G01N 33/53; G01N 33/543; C12M 1/34

(52) U.S. Cl. 435/7.1; 435/287.2; 436/518

(57) ABSTRACT

An apparatus and method for fabricating a one or two-dimensional microassays with a matrix of sites favorably disposed for screening substances such as biomolecules, chemicals or cells as described. The method includes drilling a matrix of wells or through-holes in a glass or similar material using a laser. The drilling creates a region favorably disposed towards binding a molecule or cell. A microassay plate includes a substrate and at least one hole in the substrate containing an immobilized reactant bound to an interior surface of the hole. An array of holes having chemically different immobilized reactants is provided. Holes may be drilled using one or more pulses of light of extremely short duration to create a surface in a localized area that preferentially binds to material.

### FIELD OF INVENTION

The present invention relates to apparatus and method for fabricating microassays for screening molecules or cells using lasers.

### BACKGROUND OF THE INVENTION

The worldwide pharmaceutical industry spent almost over forty billion dollars in 2001 on new drug research and development. The process of drug discovery requires sifting through hundreds of thousands of chemicals to find one drug with therapeutic potential. The techniques used to screen chemical compounds for efficacy are often tedious, labor intensive, and time consuming. As a result, the creation of new medicines is a long, risky and expensive process—taking an average of 12-13 years to turn an active substance into a marketable medicinal product.

More recently, pharmaceutical companies have been placing greater reliance on gene chips, combinatorial chemistry, cellular electrophoresis and robotics to screen compounds, rapidly and automatically, with a minimum of human intervention in the process. Of particular importance to these methodologies is the ability to screen molecules using parallel processing techniques. Microassay or microtitre plates play an enabling role in the success of the parallel processing for screening candidate compounds. Microassay plates are planar structures (e.g. a glass microscope slide or coverslip) with a matrix or array of sites on the plate's surface each of which has an affinity for a particular molecule or cell.

In one form microassay plates may be used to detect the presence of a mobile reactant that binds to an immobilized reactant affixed to the surface of the plate at a specific site. In another form the microassay plate may be used to affix cells to a feature on the microassay plate surface for subsequent detection and/or measurement using, for example, cellular electrophysiology. With respect to the former, a matrix of many differing kinds of immobilized reactants can be used to indicate the presence (or absence) of an array of mobile reactants in a sample volume of unknown composition—thereby providing a vehicle for the parallel detection of and measurement on and/or processing with specific molecules. One very well known example is the detection, measurement, and processing of nucleic acids—linear polymers in which the linked monomers are chosen from a class of four possible sub-units. In addition to being linked together to form the polymers in question, each unit has a complementary sub-unit to which it can bind. In the case of DNA, the polymers are constructed from four bases that are denoted by the letters A, T, G, and C. The bases A and T are complementary to each other, and the bases G and C are complementary to each other. When two polymers are aligned with one another and the sequences of bases are such that an A in one chain is matched to a T in the other chain and a C in one chain is matched to a G in the other chain, then the two chains will be bound together by electrostatic forces. Hence, if one segment of DNA of known order in A, T, G, and C is affixed to a known and identifiable spot on the surface of a microassay plate (the immobilized reactant), it can bind to its complementary chain (i.e. mobile reactant) if that complimentary sequence is present in a sample of unknown composition. Postprocessing using known techniques can then reveal the presence (or absence) of a complementary chain in a sample of unknown composition, provide quantitative information about the Complementary DNA, and provide samples for further processing such as amplification.

Methods to bind mobile reactant to immobilized components vary according to the particular reactant. Often times, detection is performed by tagging either the bound or mobile reactant with fluorescent or luminescent dye. Other possible identifier tags include a radioactive compound that binds to the reactant. When a fluorescent or luminescent dye is used, detection of the mobile reactant is achieved by illuminating the tagged molecule with light, thus exciting the dye to fluoresce. In these instance it is the detection of the fluorescence from the tag that proves the presence of the mobile reactant in the sample.

Medical research and/or diagnosis often involves a bank of tests in which each test requires the measurement of the binding of one particular type of mobile reactant to its corresponding immobilized reactant. Microassay plates possessing a matrix of immobilized spots that will bind to specific, known complements provide rapid, massively parallel detection of mobile reactants in a sample made up of unknown constituents. Each spot includes the immobilized component of a two-component test such as that described above, and generally a large number of different sequences are provided on a single plate. In use, the sample to be tested is brought into contact with the matrix, providing an opportunity for any mobile reactant present in the sample to bind to its complimentary, immobilized reactant known to be affixed to a specific location on the substrate. After processing, each spot in the matrix is probed to determine the presence and concentration of a mobile constituent bound to its known immobilized reactant. Microarrays have been commercialized by companies like Affymetrix, Incyte Genomics, Gene Logic, Nanogen, and Agilent.

Microarrays of the type described above are used in the detection and measurement of biomolecules that form components of a cell. In some methods of formation, the immobilized reactant is formed on the surface of a substrate either by synthesis, or deposition. In the latter case some commonly known methods of deposition include the use of a pen-nib (like an ink pen in common use before the invention of the ball-point pen) or a nozzle similar to those used in inkjet printers. Either approach is used to deposit or “spot” the immobilized reactant on the surface of the plate. In both cases it is desirable to locate the immobilized reactant in a small, well-defined and predictable location on the plate. Indeed, the smaller the spot, the closer they can be arranged on the plate and the greater the number of differing immobilized reactants that can be incorporated on its surface. As the size of a spot containing a specific immobilized reactant becomes smaller, and as more of them are placed closer together, the more difficult it becomes to prevent cross-contamination between adjacent spots containing different immobilized reactants. The consequence is a higher probability of cross-contamination and resulting false positives.

Additionally, detection and quantitative measurements require the deposition of known and predictable amounts of immobilized reactants. This is because the concentration of mobilized reactant in a sample of unknown composition will manifest itself in the intensity of the fluorescence tag. But the smaller the spot, the lower the number of immobilized reactants that can bind to a mobilized reactant, which in turn leads to lower fluorescence intensity—making it even more difficult to detect the presence of the mobile reactant.

In another form microassay plates can be use to isolate a patch of the membrane of a cell for electrophysiology studies (see for example Maher, et al, “Ion Channel Assay Methods” US Pub. US2002/0045159 A1, and U.S. Pat. No. 6,379,916 B1.) Such structures can be used for massively parallel screening of candidate drugs for use as ion channel blockers. This technique was outlined by Neher, Sakmann, and Steinback in “The Extracellular Patch Clamp, A Method For Resolving Currents Through Individual Open Channels In Biological Membranes”, Pflueger Arch. 375; 219-278, 1978 (incorporated in this patent in its entirety by reference). They found that, by pressing a pipette containing acetylcholine (ACH) against the surface of a muscle cell membrane, they could see discrete jumps in electrical current that were attributable to the opening and closing of ACH-activated ion channels. However, they were limited in their work by the fact that the electrical resistance of the seal between the glass of the pipette and the membrane (10-50 megOhms.) was very small relative to the electrical resistance of the channel (about 10 gigaOhms.). The electrical noise resulting from such a seal is inversely related to the resistance and was large enough to obscure the currents through the ion channels in the cell membrane, the conductances of which are smaller than that of the ACH channel. The large currents through the seal of the membrane to the walls of the pipette prohibit the clamping of the pipette when voltages are different from that of the bath.

It was then discovered that seals of very high resistance (1-100 gigaOhm.) could be obtained by fire polishing the glass pipettes and applying gentle suction to the interior of the pipette when brought into contact with the surface of the cell. This reduced the noise by an order of magnitude to levels at which most channels of biological interest can be studied. As a result, it was possible to greatly extend the voltage range over which these studies could be made. The improved seal has been termed a “gigaseal”. Neher and Sakmann were awarded the 1991 Nobel Prize in Physiology and Medicine for their work in developing the patch clamp technique.

Ion channels are transmembrane proteins that catalyze transport of inorganic ions across cell membranes. The ion channels participate in processes as diverse as generating and timing of action potentials, synaptic transmission, secretion of hormones, contraction of muscles, etc. Many drugs affect ion channels. Examples are antiepileptic compounds like phenytoin and lamotrigine, which block voltage dependent Na.sup.+-channels in the brain, antihypertensive drugs like nifedipine and diltiazem, which block voltage dependent Ca.sup.2+-channels in smooth muscle cells, and stimulators of insulin release like glibenclamide and tolbutamide, which block an ATP-regulated K.sup.+-channel in the pancreas. In addition to chemically induced modulation of ion-channel activity, the patch clamp technique has enabled scientists to perform voltage-dependent channel manipulations, including being able to adjust the polarity of the electrode in the patch pipette and altering the saline composition to moderate the free ion levels in the bath solution.

The patch clamp technique represents a major development in biology and medicine, since it allows measurement of ion flow through single ion channel proteins, as well as the study of the single ion channel responses to drugs. The patch-clamp technique is often referred to as “the gold standard” for drug screening and is will known to those skilled in the art. In standard practice, a thin (<1.mu.m in diameter) glass pipette is used. The tip of this patch pipette is pressed against the surface of the cell membrane. The pipette tip seals tightly to the cell and isolates a few ion channel proteins in a tiny patch of membrane. The activity of these channels can then be measured electrically (single channel recording) or, alternatively, the patch clamp can be ruptured, thereby allowing measurements of the channel activity of the entire cell membrane (whole cell recording).

The limited number of compounds that could be tested per day (typically no more than 10) has been a major obstacle to the use of the patch clamp technique as a general method in pharmacological screening of chemicals. Only one pipette can be used at a time on only one living cell at a time. The patch pipette must then be discarded. And because the mechanism of seal formation degrades with time, the pipette must be used within a few hours of the time it is made. The process is very labor-intensive, and requires a well-trained person to perform it. Several hundred thousand chemicals might have to be screened to produce one useful drug. Lastly, because this prior art processing methodology is boring, it does not excite the people with the talent needed to perform procedure day-in and day-out. Extending prior art practice of patch pipette fabrication into the planar realm is not straightforward, and several patents have been awarded and/or applied for describing inventions that attempt to address this need (representative examples include U.S. Pat. No. 6,315,940 B1 by Nisch, et al, U.S. Patent Application #US 2002/0025573 A1 by Maher, et al, all of which are included here by reference.)

There is a need for an accurate, automated method and apparatus of drug screening using parallel processing methods. Screening new drug candidates could be accelerated by a factor of as much as a 100 if an accurate, automated method was available, and companies like Axon Instruments, Molecular Devices, Nanion in Germany and CeNeS in the UK are among the few trying to address this market opportunity. There is a need to extend the prior art practice of patch pipette fabrication into the planar realm and to have a method that is more efficient and much quicker and thus less time-consuming. There is a need for an inexpensive method for fabrication of microassay plates with pits, wells or through-holes with well-defined dimensions, and possessing a known, quantifiable, and, indeed, controllable affinity for an immobilized reactant. This is especially important in cases involving extremely small dimensions and close spacing where a large number of samples are placed on a single substrate. There is also a need for improved methods of signal detection in order to deal with the unavoidable reduction in fluorescent intensity that arises as a consequence of decreasing the dimensions and increasing the density of spots on the surface. This can be achieved with improved microassay plates that include a matrix of wells or through-holes in a glass plate or functionally similar material.

This invention describes a technique that can be used to manufacture microassay plates that form a matrix of specially created through-holes that pass through the substrate to the opposite side. There is a need to drill a matrix of wells or through-holes in a glass plate or functionally similar material using a laser, but in order to drill these holes it is necessary to solve two major problems. First, when it is desirable to drill them in dielectric materials like glass it must be done in such a way that the structure of the material is not damaged (e.g. microcracks). Second the drilling process must create a region favorably disposed toward binding a molecule(s) or cell(s), and thereby provide the array of microstructures required to achieve massively parallel processing. (In the discussion that follows, “hole” is intended to refer to either a pit or well, both of which are sometimes referred to as blind holes, and through-holes, irrespective of parameters like size, shape, structure or material.)

### SUMMARY OF THE INVENTION

In accordance with this invention, an apparatus and method for creating a one or two-dimensional microassay with a matrix of sites favorably disposed for screening substances like biomolecules, chemicals or cells is described. In one embodiment, it describes a method of making the array of small diameter holes in a one or two-dimensional dielectric, such as a fiber or one or two-dimensional substrate that can be used to screen substances such as drugs in conjunction with a screening technique. In another the method includes machining holes to create localized regions whose surface is conducive to forming a bond between and the cell membrane or molecule. This apparatus and method can also be used to remove chemical or biological contaminants or cells from the environment, or capture them in space for later processing.

This invention includes drilling a matrix of wells or through-holes in a glass plate or functionally similar material using a laser that can drill holes in dielectric materials like glass without damaging the structure of the material (e.g. microcracks). It is additionally desirable if the drilling process creates a region favorably disposed toward binding a molecule(s) or cell(s), and thereby providing the array of microstructures required for massively parallel processing of many samples. In the following discussion, “receptor” is intended to refer to either a pit or well, both of which are sometimes referred to as blind holes and thru-holes, irrespective of parameters like size, shape, structure or material. The receptor can also refer to a localized region of material whose physical properties have been altered by use of a laser without the removal of material, for example, by photo-polymerization, photo-induced chemical reaction, fire polishing, the creation of locally bound static charge, or one or more combinations thereof.

In one embodiment of the present invention, the microassay plate according to the present invention includes a substrate and at least one hole in the substrate containing an immobilized reactant, the immobilized reactant being bound to the interior surface of the hole. In this embodiment, an array of holes are provided in the surface of a substrate with at least two of them having chemically different immobilized reactants that will associate themselves with different mobilized reactants. The immobilized reactants bind to mobile reactants when a solution containing the mobile reactants is brought into proximity with the immobilized reactants. The mobile and immobilized reactants may be any pair of biological or chemical compounds that have an affinity for each other. For example the reactants may be nucleic acids or antibody-antigen pairs. Another embodiment of a microassay plate according to the present invention includes a plurality of microassay holes, each hole having as surface to which a cell will bind preferentially and thus immobilize them for subsequent use.

One method for fabricating a microassay plate according to the present invention includes the steps of drilling at least one hole into a substrate using one or more pulses of light of extremely short duration. In the process, a surface is created in a localized area that preferentially binds to some other material—either a chemical that functions as an immobilized reactant or a cell. An assay utilizing an assay plate according to the present invention is carried out by bringing a solution containing a mobile reactant or cell into contact with either the immobilized reactants on the assay plate or a hole wall. A molecular assay plate is then washed to remove unbound material. The amount of mobile reactant bound to the washed assay plate is then determined using techniques well known to those skilled in the art.

In another embodiment the hole is constructed in a manner that provides an affinity for a biological or chemical molecule, or cell to immobilize or affix it at a known location for further analysis—the affinity being created by localized photo-polymerization, photo-induced chemical reaction, photo-induced emission, laser induced fire polishing, leaving behind positive ions frozen in the surface that provide an electrostatic binding force to other substances, creating a region that is locally hydrophilic on a surface that is otherwise hydrophobic, or the like.

In yet another embodiment, one or more pulses of light are used to drill a shaped through-hole for use in single or whole cell patch-clamp experiments.

In yet another embodiment, the walls of the hole may be structurally modified by the controlled deposition of heat to the surface.

In yet another embodiment, the deposition of heat is provided by means of a burst of short pulse pulses.

In yet another embodiment, the deposition of heat is provided by means of a separate laser with emission at a separate wavelength.

In yet another embodiment, the deposition of heat is provide by the same laser running in two different pulse width regimes, the first resulting in ablation or structural modification of a portion of the substrate predominantly through plasma formation and the second structurally modifying the structure of the material by depositing heat in the material.

In yet another embodiment the deposition of heat is provided by providing a ultrashort pulse of light generated by a source that also provides pulses of longer duration at the same or different central wavelength of operation.

In yet another embodiment the structural modification may occur as a result of elevating the temperature of the substrate to a level that is below the melting temperature.

In yet another embodiment, a waveguide is written in the substrate to optically connect the blind hole containing the immobilized reactant to a surface, thereby providing a conduit for fluorescent light to be “piped” to a point in close proximity to a detector.

In still another embodiment, the substrate includes a dopant that absorbs light at the wavelength of excitation of the fluorescent or luminescent tag and transmits at the wavelength of fluorescence.

In still another embodiment, excitation of the fluorescent tag is provided by absorption of at least 2 photons of fundamental wavelength.

In still another embodiment, the excitation of the fluorescent tag is provided by absorption of two or more photons of a fundamental wavelength and the detector that detects the fluorescence is blind at the fundamental wavelength of excitation.

In still another embodiment, excitation of the fluorescent tag is by a pulsed laser source and detection is gated in time to so that the detector is open to receiving a signal from the fluorescent tag during a specific time interval.

In still another embodiment reflecting surfaces are provided to reduce noise created by transmission of the fluorescence excitation wavelength to the detector.

In still another embodiment, means are provided to control the affinity for binding of a chemical, biomolecule or cell membrane to the inner surface of the hole or receptor.

In still another embodiment, means are provided to preserve the affinity for binding to the hole wall.

In yet another embodiment of the invention each substrate or section thereof is marked with one or more, single or two-dimensional codes (numbers bar codes, etc.) designed to uniquely identify each substrate or subset of the matrix thereof.

### DETAILED BRIEF DESCRIPTION OF THE DRAWINGS

In the detailed description of the preferred embodiments of the invention presented below, reference is made to the accompanying drawings.

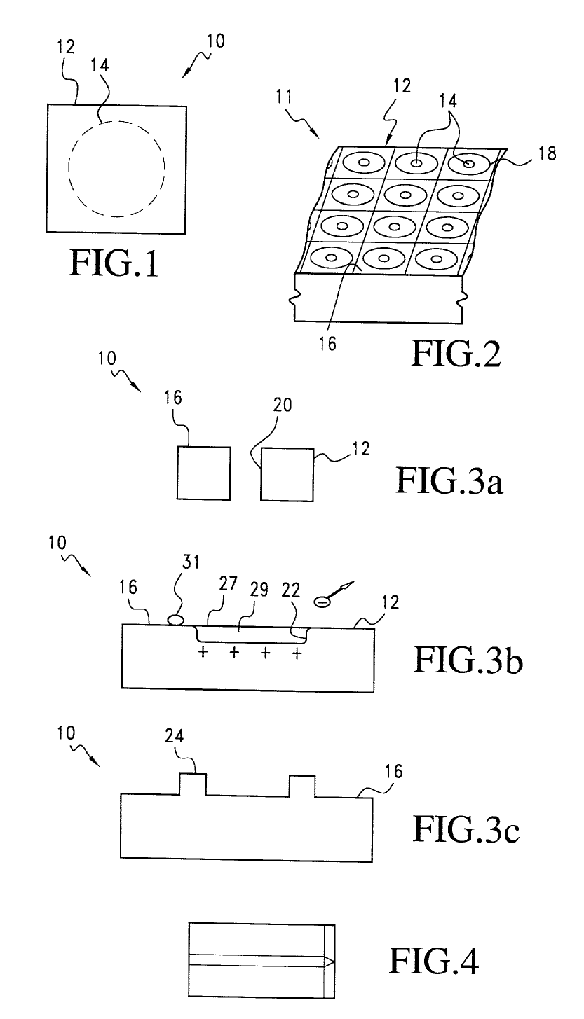

FIG. 1 shows a schematic of a microassay plate with a receptor.

FIG. 2 shows a schematic of a microassay plate with an array of receptors.

FIG. 3 shows portions of a microassay plate with a through hole, a blind hole and a raised wall that may have a continuous wall.

FIG. 4 shows a schematic of a portion of a microassay plate an intermittent wall fabricated as a channel.

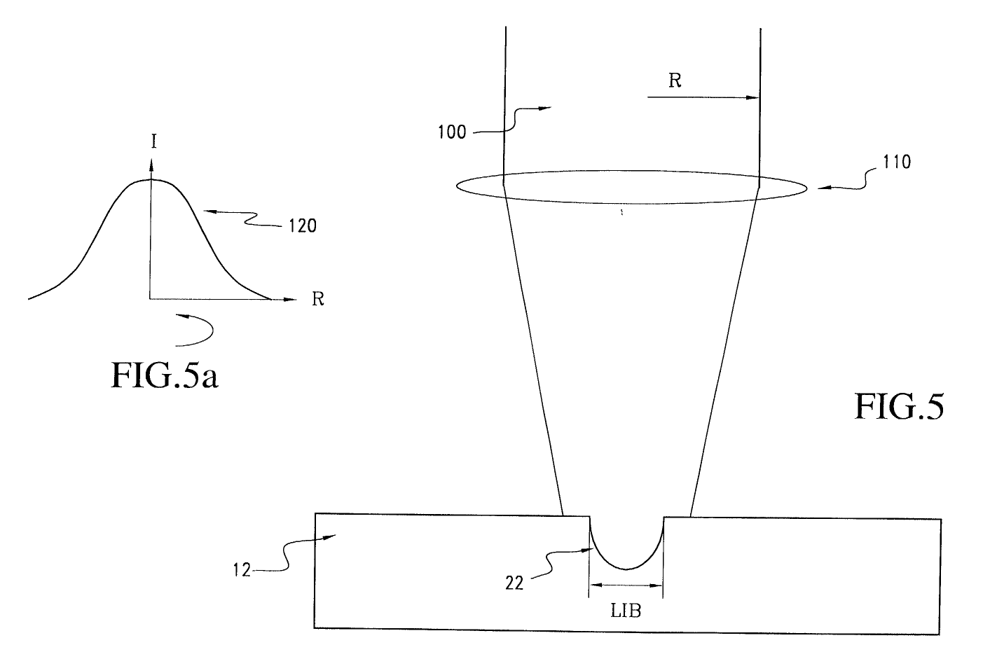

FIG. 5 shows a schematic of the fabrication of a microassay plate including the method for creating receptors in the surface of a material.

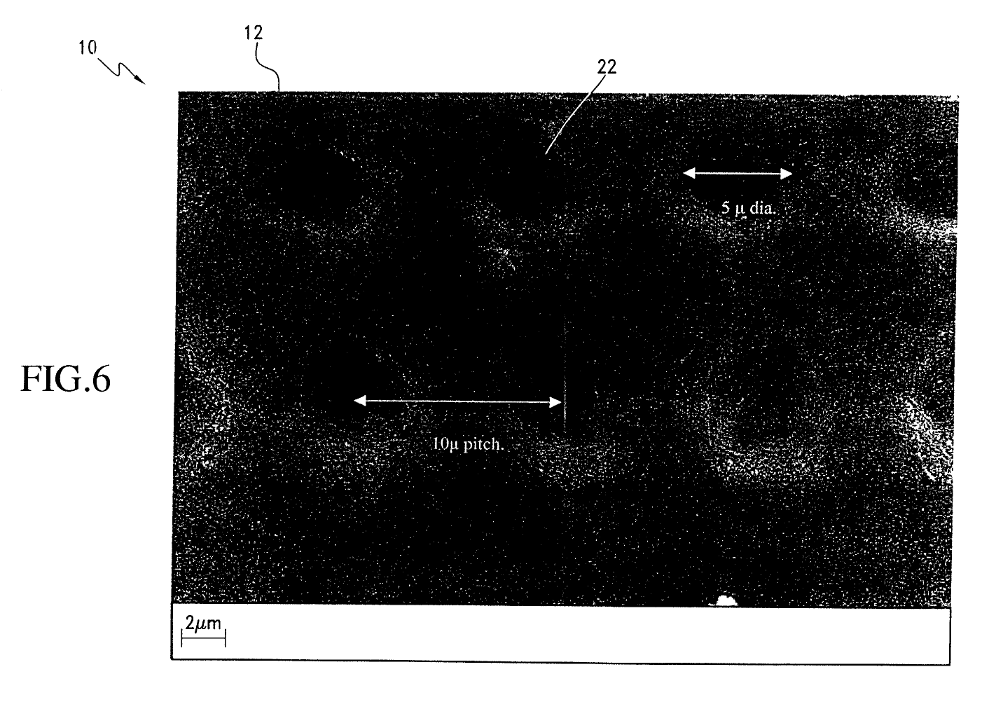

FIG. 6 is a characterization taken from a SEM photograph of how the method of fabrication of these holes illustrated in FIG. 5 creates pits in the surface with an affinity for binding to a biomolecule or cell.

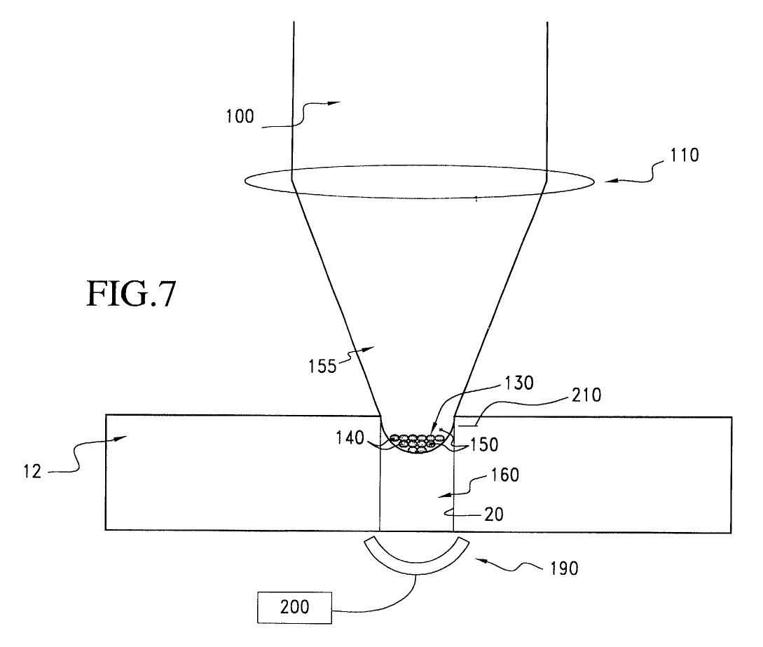

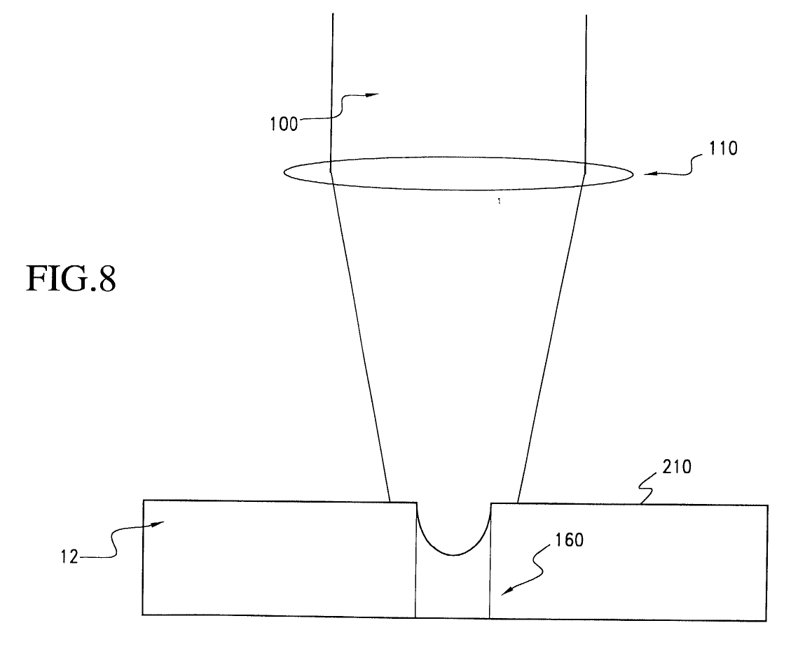

FIG. 7 illustrates how a series of blind holes created in the surface of glass using the present method can be confined with waveguides directly written in the substrate to channel fluorescent light to the back surface for detection.

FIG. 8 is defective and needs to be removed.

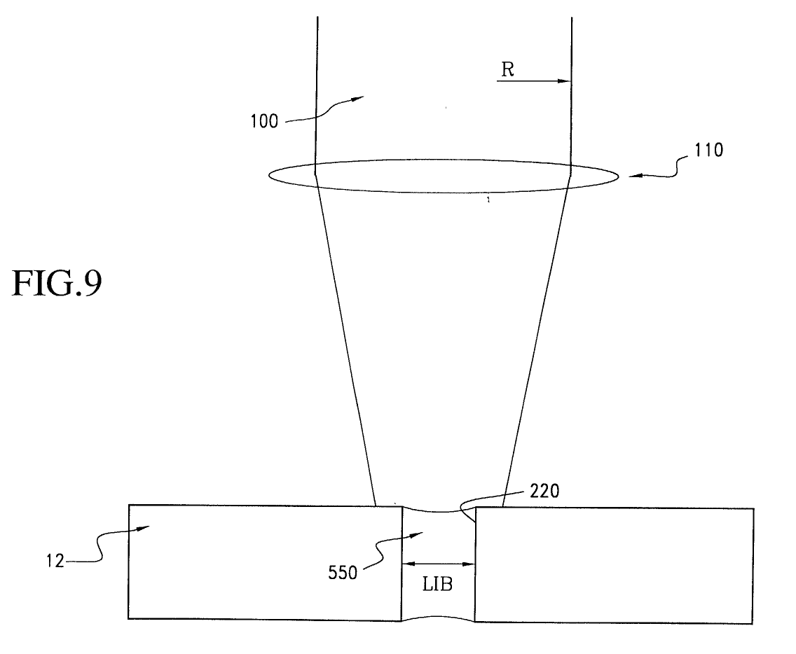

FIG. 9 shows how the method operationally described in FIG. 5 can also be used to machine a through-hole in the substrate.

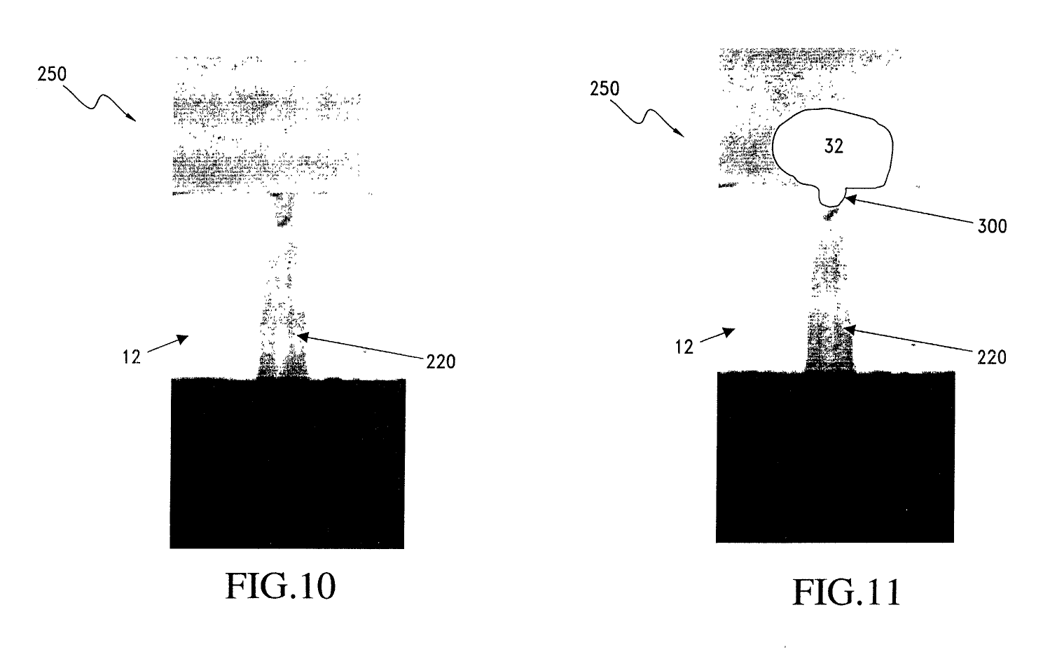

FIG. 10 is a picture of a through-hole machined in glass according to the present invention.

FIG. 11 illustrates how a cell would be attached to the hole for electrophysiology measurements.

### DETAILED DESCRIPTION

The present description will be directed in particular to elements forming a part of, or in cooperation more directly with, the apparatus in accordance with this invention. It is understood that elements not specifically shown or described may take various forms well known to those skilled in the art. Referring now to the drawings, where like reference numerals represent similar or corresponding parts throughout several views.

FIG. 1 is a schematic of a screening apparatus 10, also known as a microassay plate 10, fabricated from a solid substrate, possibly a dielectric like glass 12 or a semiconductor. The screening substrate or apparatus 10 has one or more particle attracting receptors 14. FIG. 2 shows an array of receptors 14, each receptor in the surface 16 of the dielectric, and a surrounding area referred to as a peripheral wall 18. The peripheral wall 18 can be part of a through hole 20 (FIG. 3a) or blind hole 22 (FIG. 3b) set in the surface 16 or it can be part of a raised area 24 (FIG. 3c) above the surface 16 as shown in FIG. 3. The peripheral wall 18 can be a continuous wall 26 or an intermittent wall 28 as shown in FIG. 4 as a channel. FIG. 3 also shows that the receptor can be formed by creating a positively charged region 27 that may be in an ablated area.

The microassay plate 11 is used with a number of liquid samples comprising respectively different substances, such as proteins, in the holes in the array. These proteins form the immobilized reactant 29 also known as chemical probes shown in FIG. 3. A idetification molecule is an example of a chemical probe. A second fluid 31 containing particles that are to be analyzed contacts the chemical probe 29 for a predetermined period of time so that particles in the fluids may have time to interact (e.g., bind, react) with the proteins on the microarray surface. After the predetermined time has elapsed, the microarray may be washed and/or exposed to a wash or reagent liquids to remove any unbound particles or reaction products. The wash and/or reagent liquids can address each hole independently or jointly, or through exposure to a liquid source like flooding. The microarray surfaces can then be analyzed to determine which, if any, of the particles may have interacted with the immobilized reactants 29. The receptor may actually include both a substrate material 12 and the immobolized reactant 29.

One way of fabricating the screening substrate apparatus 10 according to the present invention begins by focusing a beam of light consisting of one or more pulses of ultrashort duration (less than about 100 ps) generated by the ultrashort pulse laser onto the glass substrate 12. The intensity of the beam of light might be arranged so that it is sufficient to induce multiphoton effects like multiphoton absorption over dimensions less than or comparable to the full size of the beam. The result is a rapid increase in temperature of the material. There are two threshold effects. The first, lower threshold, induces a change in the physical structure of the material without removal of a substantial portion. The second, higher threshold, results in absorption in a very thin layer of the material creating a plasma which then expands away from it, with the lighter electrons in the plasma ejected more rapidly than the positively charged, heavier ions. Some of these ions will be trapped on the surface 16 creating an attractive region 30.

The attractive spot 30 is favorably disposed towards electrostatically binding a particle 32 such as a molecule or cell. It is important to recognize that this method of creating a localized spot on the surface of the material possessing an affinity for molecules or cells can be accomplished without actually removing any substantial amounts of matter. The rapid deposition of heat confined to an area on the surface of the material whose dimensions are less than the full size of the beam incident on it. Multiphoton absorption can result in a burst of electrons being emitted from the surface, leaving a positively charged region behind that rapidly cools, freezing ions therein. These embedded ions then serve as an attractive binding force for chemicals, biomolecules, and cell membranes.

FIG. 4 is a schematic illustration of a beam of light 100 being focused by a lens 110 onto the solid substrate 12. More particularly, the beam of light consists of one or more pulses with a particular duration more fully described in U.S. Pat. No. 5,656,186 and/or US patent application 2001/0009250 by Herman, et al (incorporated herein in their entirety including reissues and pending divisionals). FIG. 5 is a graph of the intensity of the pulse of light (along the horizontal axis of the figure) varying with time (along the vertical axis of the figure). In FIG. 5 curve 120 is illustrates that the intensity, I, of the light beam 100 varies as a function of radius, R. Those skilled in the art will recognize that this is but one of many beam intensity profiles that could be used in this invention. The choice of this particular profile is intended only for illustration and is not intended to be limiting.

If the light beam 100 intensity is sufficiently high, then it will exceed the threshold for laser induced breakdown (LIB) of the material of solid substrate 12 and some of the material will be ablated (or modified) to create the receptor, sometimes referred to and illustrated as the blind-hole 22. Laser induced breakdown (LIB) can also result in alteration of the physical structure of the material without its removal as, for example, by melting and resolidification, or inducing a chemical reaction with nearby liquid or gaseous atmospheres all of which can create receptors of different types, as will be further discussed below. With each pulse of light 100 incident on substrate 12 some material will be removed or modified. If desirable to do so, a hole can be drilled into the substrate by the use of one or more pulses of light in this manner. Clearly, either blind holes (as further discussed below) or through-holes can be fabricated in this manner and even raised peripheral walls 18.

The shape for the holes can be tailored by employing processing methods such as trepanning as is well known in the art. As described in U.S. Pat. No. 5,656,186, reissues, and divisionals, a key benefit of LIB is that the process is highly deterministic. Virtually identical results are obtained regardless of the number of free electrons trapped in the surface of material onto which the beam of light is directed. As a result, it is possible to induce LIB over dimensions much smaller than the diffraction-limited spot size of the beam, and alter or remove material in an extremely precise and predictable manner. Another advantage of LIB is that the deposition of energy occurs on a time scale that is short compared to the time it takes for a significant amount of energy in the form of heat to propagate into and damage material adjacent to the LIB zone which will become the receptor, thus reducing and even eliminating deleterious effects on the substrate.

When removing material, as shown in FIG. 3, the LIB method described herein involves the creation of a plasma that expands away from the substrate with the lighter, more volatile electrons coming off first followed by a cloud of heavier, less volatile positively charged ions It is the residual positive ions that are trapped in the material left behind which important to fabricating the screening apparatus 10 of the current invention. At the same time, the boundary between the LIB material and the solid structure is not discontinuous—some heat from the plasma may flow into a very thin layer of adjacent material sufficient to create a layer that traps some positive ions when the material cools. In fact it may be desirous to facilitate this process by intentionally depositing heat locally in the ablated area, either by employing a burst of ultrafast laser pulses, by using somewhat longer pulses to allow time for some flow of heat into the surrounding material, and/or by providing combinations of ultrashort and not so ultrashort pulses to the material to achieve the desired result. These imbedded positive ions provide an immobilizing electrostatic attraction for binding molecules and/or cell membranes to the surface in which they are embedded. This is the force that creates the binding affinity localized to the zone of LIB, herein known as the receptor 14.

FIG. 6 represents a SEM photograph of an embodiment of the screening substrate apparatus 10 of the current invention where the receptor are, in this case, blind-holes 22, formed in the surface of the glass substrate 12 using an ultrashort pulsed laser (in this case a Clark-MXR, Inc. Model CPA-2001 Ti:Sapphire Regenerative Amplifier) in a ultrafast laser micromachining workstation (illustratively, the Model RS-2001 Ultrafast Micromachining Workstation manufactured by Clark-MXR, Inc.) The blind-holes are approximately 5 microns in diameter at the surface and are spaced at about 10 microns. Each one of these blind-holes will have positive charge imbedded in the surface of the wall, creating a localized affinity for biomolecules, cell membranes, and the like. Since the screening substrate apparatus 10 has one or more receptors 14 with this positively charged surface 30, it is favorably disposed towards holding the particle, such as the molecule or cell, because it is capable of forming the attractive bond 32 with the particle.

A matrix of 2540 by 2540 holes 20 in a glass substrate 12, as shown in FIG. 6, would enable the localization of over 6 million different immobilized reactants on an area the size of one square inch (both larger and smaller dimensions are within the capabilities of the manufacturing process, and actual dimensions may vary depending upon the need). The matrix of holes 20 of this dimension can be produced rapidly and repeatedly using this technology, allowing for mass production at low cost. Use of a microassay for testing is well known in the literature (see for example, US Patent Applications 2002/0045169 A1, 2002/0048754 A1, incorporated herein by reference in its entirety).

The screening substrate apparatus 10 shown in FIG. 7 can be designed and fabricated to incorporate means of enhancing the signal that comes from a fluorescent tag associated with a bound mobile reactant 140 while minimizing background noise created by, for example, the light used to excite the fluophor. To this end the same ultrashort pulse light source (or, if desired, a different one) can be used to direct write a waveguide channel between the hole created in a first surface and that of a second surface arranged in close proximity with a detector. The direct writing of waveguides inside materials using ultrashort pulses of light is being commercialized by companies like Translume—www.translume.com and is as illustrated in FIG. 7. In FIG. 7, a mobile reactant 140 bound to an immobile reactant 130 at receptor 14, fabricated as described above, is tagged with a fluorescent molecule 150. Upon illumination with an excitation beam 155 at a wavelength at which the fluorescent molecules absorbs, fluorescent light will be emitted in all directions. A large fraction of that emission will be channeled down the waveguide 160, to the detector 190 for recording by electronics 200. In this manner the intensity of the fluorescent light will be enhanced at the detector, thereby improving the strength of the detected signal. The apparatus may also include indicia 210 formed by the same process. These indicia can be a bar code such as a two dimensional bar code.

In an array of holes, as shown in FIG. 2, the surface of a substrate like glass can be optically connected to individual waveguides written in the glass substrate so as to channel the light from each hole to a detector on the other side. Indeed, the direct write manufacturing process of this invention is flexible enough so that the spacing and diameter of each hole and its associated waveguide can be fabricated so that they line up with or match individually addressable elements of an array detector such as those commonly found in CCD cameras. In the detection process the microassay plate would be positioned with respect to the array detector using fiducials written into the substrate itself for that purpose and the same laser source can be employed for that purpose. Or reference patterns consisting of two or more array spots may be imbedded in the matrix itself to serve as both a reference test pattern to calibrate signal strength and serve as an alignment reference marks. Another benefit of this embodiment is that there would be less crosstalk between adjacent detection elements (See for example US Patent Application 2002/0004204 A1 incorporated herein by reference for alternative embodiments.)

In yet another embodiment of the present invention, the substrate itself might be a linear array of spots machined in a glass fiber or ribbon as more fully described in US Patent Application No. 2001/0051714 A1. Here only one detector would be required, and the linear array would move under a stationary excitation source and detector.

In yet another embodiment of the present invention, one could choose to fabricate the microassay out of material that absorbs at the excitation wavelength and is at least nominally transparent at the wavelength at which the fluorophore tag fluoresces. Substrates possessing this characteristic are known to those skilled in the art as blocking, interference, dichroic, or shoulder filters. The beneficial aspect of using such a material is that the excitation wavelength would be absorbed before reaching the detector, reducing or eliminating detector noise and improving signal delectability.

In an additional embodiment, one or more surfaces of the substrate 12 can be coated with a filter-block coating 210 to absorb or reflect the excitation wavelength before manufacture. In still another embodiment reflecting surfaces can be provided to reduce noise created by transmission of the fluorescence excitation wavelength to the detector.

The process of drilling the holes in the surface would also drill through this layer, as shown in FIG. 8. Alternatively, or in addition, a layer of hydrophobic material may be uniformly deposited on the surface of the substrate to create the coating 210 before machining, thereby providing yet greater localization of the immobilized reactant 130 to the receptors 14 drilled in the surface of the substrate 12.

In still another embodiment of the present invention, the substrate itself, or an additive to the sample that characteristically does not interfere with the hybridization process, can be employed as an absorber of light at a wavelength other than that employed for excitation or detection of the mobile reactant. When illuminated at this wavelength, the absorption of light in the substrate or additive will heat the sample locally, facilitating the hybridization process. This concept can be extended to include direct photo-excitation of either the immobilized or mobile reactant in order to facilitate binding of the two through absorption of multiple photons at the appropriate wavelengths.

In still another embodiment, the fluorescent excitation wavelength may be at a harmonic of the sources fundamental wavelength and excitation of the fluorophore occurs as a result of multiphoton absorption. The use of such a source (especially one generating ultrafast pulses of high repetition rate) can be favorable if the fluorescence detector is chosen so that it is blind at the fundamental wavelength of excitation.

In yet another embodiment of the present invention, a second beam of light is chosen to have a wavelength that is absorbed by the substrate is directed onto the surface of the machined hole in order to create a thin melt layer which, when solidified, provides a mechanically and optically smooth surface to which an immobilized reactant or cell will affix. This second beam is chosen to have a pulse duration substantially longer than that use to create the hole itself, or could arise from the use of a burst of many ultrashort pulses closely spaced in time similar to those described in US Patent Application 20010009250 incorporated herein in its entirety by reference. A representative example of a second source that might be used to create a thin, surface melt layer that resolidifies into a smooth surface, one might use a pulsed CO2 laser of chosen pulse duration and energy to create the melt without depositing so much energy into the material that it creates a heat-affected zone (see for example disclosure xxxxx incorporated herein by reference.)

Deposition of immobilized reactants (probes) are well known in the literature (see for example US Patent Application 2001/0036674 A1) and are included here in their entirety by reference. Detection means are also well known in the literature. Representative examples of prior art that might make use of this invention include (but are not limited to) U.S. Pat. No. 6,025,129, US Patent Applications 2002/0040275 A1, 2002/0006604 A1, and 2002/0004204 A1.

Microassays for Use in Cellular Electrophysiology

Referring now to FIG. 9 we see a schematic illustration of the micromachining method machining a through-hole 220, the hole itself is machined through the substrate to the back surface. This through-hole characteristically has positive ions 550 embedded in its surface forming a layer favorable to binding to other substances like the membrane of a cell. When fabricated with proper dimensionality (e.g. sub-micron diameter at the top surface) such a hole can serve a function similar to that of a patch pipette used to isolate a single or few in number ion channels in the cell membrane. Or when fabricated with somewhat larger dimensionality (e.g. 1 to 3 micron diameter at the top surface) as a patch pipette for whole cell recording. In either case the formation of a so-called gigaseal is advantageous, and the particular use of ultrafast lasers to machine these holes is beneficial in this regard. The additional employment of heat deposited in a thin layer on the surface of the through-hole by use of a long pulse of light burst of pulses or a secondary, longer pulse as previously described will serve to create a mechanically smooth wall to which the cell membrane can adhere. Laser processing in this manner treats the surface of the substrate in a manner similar to the flame polishing of the pipette that is standard practice in single pipette patch clamp methodologies. It is not clear that additional smoothing of the surface is required to form a gigaseal, and the invention described herein is not intended to be limited by the mechanical shape of the surface.

FIG. 10 is a picture of a through-hole 220 in a glass substrate 12 that serves as an illustrative example of the screening apparatus 250. FIG. 11 shows how the particle 32, such as a cell or molecule, is drawn into the through-hole 220 so that its membrane forms a seal 300 with the top and/or side walls of the hole. An array of structures fabricated in this manner, when employed in conjunction with a method involving holding the cell and applying a light vacuum, for example, until bursting the cell membrane that is called a whole-cell, patch-clamp type method, known to those in the art. Typical methods for placing the cell in communication with the hole are known to artisan in the field. Representative examples can be found in U.S. Pat. Nos. 6,127,133, 6,117,291, 6,063,260, and 6,287,758.

Several ways of creating this array have been described in the above text. Others are possible. It is expected that this drilling process may have to be done in such a way that the electrostatic charge created by the trapped positive ions is preserved for transportation and later use. This invention includes methods to prevent contamination of the surface. Holes can be machined in a vacuum or in a specialized atmosphere free of the contaminants that would degrade the efficacy of the gigaseal between the time the hole is drilled and when it is used. It is also possible to package the end product in an environment free of contaminants that can degrade formation of the gigaseal.

Another variation on this invention includes machining the receptors, including holes and channels, in the presence of an electric field as more fully described in Invention Disclosure 451162/502132 cited above and filed with the USPTO Document Disclosure Feb. 2, 1999 and refilled again on Dec. 13, 2001 (incorporated as part of this disclosure by reference.) The presence of this electric field during the ablation process would accelerate electrons away from the material while at the same time causing the positively charged ions to come off more slowly. The result would be more positively charged ions trapped in or on the surface of the material left behind. Yet another variation on this invention is to heat the entire substrate to a temperature just below its melting point before machining it with light pulses of any duration. The effect of this preheating would be to reduce the amount of energy that needs to be transferred to the surrounding material from the plasma in order to create the taffy or melt layer of material in which the positively charged ions are trapped.

Embodiments of the invention may be used in any number of different fields. For example, embodiments of the invention may be used in pharmaceutical applications such as proteomic or similar studies for target discovery and/or validation as well as in diagnostics in a clinical setting for staging or disease progression. Also, embodiments of the invention may be used in environmental analyses for tracking and the identification of contaminants. In academic research environments, embodiments of the invention may be used in biological or medical research. Embodiments of the invention may also be used with research and clinical microassay systems and devices for drug screening, nucleic acid sequencing, mutation analysis, gene expression, fingerprinting, forensic analysis, and the like.

In embodiments of the invention, events such as binding, binding inhibition, reacting, or catalysis between two or more components can be analyzed. For example, the interaction between an analyte in a liquid sample and a capture agent bound to a surface may be analyzed using embodiments of the invention. More specifically, interactions between the following components may be analyzed using embodiments of the invention: antibody/antigen, antibody/hapten, enzyme/substrate, carrier protein/substrate, lectin/carbohydrate, receptor/hormone, receptor/effector, protein/DNA, protein/RNA, repressor/inducer, DNA/DNA and the like.

Samples may be derived from biological fluids such as blood and urine or from gases in an atmosphere suspected of contamination with known or unknown pathogens. In some embodiments, the biological fluids may include organelles such as cells or molecules such as proteins and nucleic acid strands. When the microassay plate is used to analyze, produce, or process a biological fluid or a biological molecule, the chip may be referred to as a “biochip”.

The liquids may be provided by a disperser and may comprise any suitable liquid media and any suitable components. Suitable components may include analytes, capture agents (e.g., immobilized targets), and reactants. Suitable analytes or capture agents may be organic or inorganic in nature, and may be biological molecules such as polypeptides, DNA, RNA, mRNA, antibodies, antigens, etc. Other suitable analytes may be chemical compounds that may be potential candidate drugs. Reactants may include reagents that can react with other components on the sample surfaces. Suitable reagents may include biological or chemical entities that can process components at the sample surfaces. For instance, a reagent may be an enzyme or other substance that can unfold, cleave, or derivatize the proteins at the sample surface. Suitable liquid media include solutions such as buffers (e.g., acidic, neutral, basic), water, organic solvents, etc.

In an illustrative example of how a microassay plate according to an embodiment of the invention can be used, a first dispenser may deposit a number of liquid samples comprising respectively different proteins in the holes in the base of the chip that form the immobilized reactant also known as chemical probes. The first dispenser may be a “passive valve” type dispenser. Passive valve type dispensers are described in further detail below. A second dispenser, which may be the same or different than the first dispenser, can then dispense fluids comprising analytes into the holes. The fluids may remain in contact for a predetermined period of time so that analytes in the fluids may have time to interact (e.g., bind, react) with the proteins on the sample surfaces. After the predetermined time has elapsed, the sample may be washed and/or exposed to wash or reagent liquids to remove any unbound analytes or reaction products. The wash and/or reagent liquids can address each hole independently or jointly, or by exposure to a liquid source through, for example, flooding. The sample surfaces can then be analyzed to determine which, if any, of the analytes in the fluids may have interacted with the bound proteins (immobilized reactants).

The analysis may take place using any suitable process and may be quantitative or qualitative. The sample surfaces may be analyzed to determine, for example, which analytes bind to the sample surfaces and/or how many analytes are bound to the sample surfaces. In one embodiment, fluorescent tags can be attached to the analytes in the fluids, while the proteins bound to the sample surfaces are free of tags or contain different tags. Binding between the analytes and the bound proteins can be observed or detected by, for example, fluorescence, chemiluminescence, fluorescence polarization, surface plasmon resonance (SPR), imaging SPR, ellipsometry, or imaging ellipsometry, chromogenic labels, and other spectroscopic means (Raman, etc.).

In another example of how the chips according to embodiments of the invention may be used, potential drug candidates and a plurality of potential drug candidates can be assayed almost simultaneously. For instance, synthesized organic compounds may be tested for their ability to act as inhibitors to a family of receptors that are immobilized at different sample locations. The synthesized compounds and binding ligands for the receptors may be present in liquid samples that are deposited in the wells of a chip. Receptors corresponding to the ligands may be immobilized therein. After the liquid samples are deposited in the wells, a period of time may then pass to allow any potential interactions to occur between the ligands and the receptors. The sample surfaces may then be analyzed to see if the ligands bind to the receptors. If a binding ligand in a liquid sample does not bind to the immobilized receptor, the organic compound dispensed with the ligand may inhibit the interaction between the ligand and the receptor. The organic compound may then be identified as a potential drug candidate.

In another example, liquid samples containing proteins may be deposited in the wells of a chip. When the sample surfaces receive the liquid samples, they may be within or proximate to the fluid channels of a dispenser. At this point, each fluid channel can serve as a reaction chamber where a reaction can take place. For example, while the sample surfaces of the chip are within or proximate to the fluid channels, various other reagents in liquid samples may be deposited on the previously deposited samples. The reagents can unfold, cleave, or derivatize the proteins in the previously deposited liquid samples. The proteins in the liquid samples may be processed while they are (1) on the sample surfaces, (2) in liquid drops on the sample surfaces, or (3) while the sample surfaces are in or proximate to the fluid channels. The processed proteins may then be transferred to an analysis device such as a mass spectrometer. In other embodiments, proteins in the deposited liquid samples may, for example, unfold or cleave without subsequently deposited reagents. For example, the proteins in deposited liquid samples may unfold, cleave, or otherwise change if left on the sample surfaces for a predetermined period of time.

Although proteins are mentioned in this example and in other examples, other compounds could serve as a reactant, a catalyst, or an enzyme. A component that is bound to a well may be a counterpart to the reactant, catalyst, or enzyme. It is understood that proteins are cited herein as exemplary samples and components and embodiments of the invention are not limited to the processing or analysis of proteins. In embodiments of the invention, the interaction between any two components may be analyzed.

The invention has been described in detail with particular reference to certain preferred embodiments thereof. It will be understood that variations, combinations, and modifications can be affected within the spirit and scope of the present invention.

1. A substrate for selectively attracting particles comprising:
a surface;
a particle attracting receptor adjacent to the surface; and
at least a portion of the receptor having a positive charge.

2. The substrate of claim 1 wherein the receptor is a through-hole.

3. The substrate of claim 1 wherein the receptor is a blind hole.

4. The substrate of claim 1 wherein the receptor comprises a peripheral wall extending above the surface.

5. The substrate of claim 1 wherein the receptor comprises a region on the surface of the substrate.

6. The substrate of claim 1 comprising a plurality of receptors.

7. The substrate of claim 1 in which the substrate comprises identification indicia.

8. The substrate of claim 7 wherein the identification indicia is formed by the same process that forms at least one receptor.

9. The substrate of claim 7 wherein the indicia comprises a bar code.

10. The substrate of claim 9 in which the bar code comprises a two dimensional bar code.

11. The substrate of claim 1 wherein the substrate comprises a glass.

12. The substrate of claim 1 in which the substrate comprises a semiconductor.

13. The substrate of claim 7 comprising identification indicia formed on the surface.

14. The substrate of claim 7 comprising indentification indicia formed in the substrate.

15. The substrate of claim 1 comprising a plurality of receptors arranged along a channel.

16. The substrate of claim 1 comprising a plurality of receptors arranged in an array.

17. The substrate of claim 1 in which the receptor comprises an immobilized reactant.

18. The substrate of claim 17 wherein the immobilized reactant comprises at least one molecule.

19. The substrate of claim 17 in which the immobilized reactant comprises at least one molecule that fluoresces upon excitation when attached to a specific particle.

20. The substrate of claim 17 wherein the reactant comprises a tag that absorbs light at a wavelength of excitation and emits light at a characteristic wavelength different from the wavelength of excitation.

21. The substrate of claim 1 wherein the surface comprises a reflecting surface.

22. The substrate of claim 21 wherein the reflecting surface reflects light at an excitation wavelength.

23. The substrate of claim 1 comprising a filter on the surface to filter out the excitation wavelength.

24. The substrate of claim 1 wherein the substrate comprises a colored glass.

25. The substrate of claim 23 wherein the filter comprises a coating on the surface.

26. The substrate of claim 1 in which at least a portion of the surface surrounding a receptor is hydrophobic or liquiphobic.

27. The substrate of claim 1 comprising a coating on at least a portion of the surface that is resistant to binding of an immobilized reactant.

28. The substrate of claim 27 in which the coating comprises alkane thiols or polyethylene glycol.

29. The substrate of claim 24 in which at least a portion of the surface surrounding the immobilized reactant comprises a hydrophobic or liquiphobic material.

30. The substrate of claim 24 comprising a coating on at least a portion of the surface surrounding the immobilized reactant that is resistant to binding of an immobilized reactant.

31. The substrate of claim 32 comprising a binding means for controlling the binding of a cell membrane to the receptor.

32. The substrate of claim 1, wherein the particle attracting receptor comprises a cell attracting receptor.

33. The substrate of claim 1, wherein the particle attracting receptor comprises at least one molecule attracting receptor.

34. The substrate of claim 33 comprising binding means for controlling the binding of at least one molecule to the receptor.

35. The substrate of claim 20 wherein the tag comprises a luminescent or radioactive tag.

36. The substrate of claim 20 wherein the tag comprises a luminescent tag and the tag emits at a luminescent wavelength.

37. The substrate of claim 20 in which the fluorescent tag is characterized by multiphoton absorption.

38. The substrate of claim 1 comprising at least one waveguide coupled to at least one receptor.

39. The substrate of claim 38 in which the waveguide is coupled to a surface other than the one to which the receptor is affixed.

40. The substrate of claim 38 wherein the waveguide optically connects the receptor to a surface other than the one on which the receptor is affixed.

41. A method for fabricating a microassay plate comprising: directing at least one pulse of light having a pulse width less than 100 ps from a first laser to at least one localized area on the surface of the plate to form a receptor.

42. The method of claim 41 wherein directing at least one pulse of light comprises ablating a portion of the surface to create the receptor.

43. The method of claim 41 comprising heating the surface.

44. The method of claim 43 comprising melting and resolidifying at least a portion of the surface to form the receptor.

45. The method of claim 44, the heating step comprising heating the surface with a burst of light pulses.

46. The method of claim 44, comprising melting and resolidifying using a laser. 47. The method of claim 43, in which the heating step comprises heating the surface to a temperature less than that required to form a plasma.

47. The method of claim 43, in which the heating step comprises heating the substrate to a softening temperature that is below the melting temperature.

48. The method of claim 47, in which the heating step comprises heating the surface with a laser.

49. The method of claim 41 comprising directing a beam of light pulses on the surface the pulses characterized by a pulse length sufficient to create positive ions, ablate the surface, and heat the surface adjacent to the ablated site to form a liquid layer.

50. The method of claim 41 comprising forming the receptors in a vacuum.

51. The method of claim 41 comprising forming the receptors in an atmosphere free of contaminants.

52. The method of claim 41 comprising packaging the assay in an environment free of contaminants.

53. The method of claim 41 comprising forming the receptors in the presence of an electric field.

54. The method of claim 41 comprising heating the micro assay plate to a temperature just below its melting point.

55. The method of claim 49 comprising preheating the micro assay plate.

56. The method of claim 41 comprising forming the receptors in a body of glass characterized by a characteristic filtering wavelength.

57. The method of claim 41 comprising coating the surface with a layer of filter material.

58. The method of claim 41 comprising coating the surface with a layer of a reflective material.

59. The method of claim 41 comprising writing waveguide in the plate.

60. The method of claim 59 comprising forming the waveguide and the receptor with a single laser.

61. The method of claim 41,comprising coating at least a portion of the surface with a hydrophobic or liquiphobic material prior to forming the receptor.

62. The method of claim 49 comprising coating at least a portion of the surface with a hydrophobic or liquiphobic material prior to forming the receptor.

63. The method of claim 59 comprising coating at least a portion of the surface with a hydrophobic or liquiphobic material prior to forming the receptor.

64. The method of claim 41 comprising a coating at least a portion of the surface with a material that is resistant to binding of an immobilized reactant prior to forming the receptor.

65. The method of claim 49 comprising a coating at least a portion of the surface with a material that is resistant to binding of an immobilized reactant prior to forming the receptor.

66. The method of claim 59 comprising a coating at least a portion of the surface with a material that is resistant to binding of an immobilized reactant prior to forming the receptor.

67. The method of claim 64 comprising coating the surface with alkane thiols or polyethylene glycol.

68. A method of making a substrate favorably disposed towards holding a cell comprising:
providing a substrate having a surface;
directing at least one pulse of light having a pulse width less than 100 ps from a first laser to at least one localized area on the surface of the plate to form a receptor area, void, or hole characterized by an affinity for a cell.

69. The method of claim 68 wherein directing at least one pulse of light comprises ablating a portion of the surface.

70. The method of claim 68 comprising heating the surface during the step of directing a beam of less than 100 ps pulses of light on the surface of the substrate.

71. The method of claim 68 comprising melting and resolidifying at least a portion of the surface of the receptor.

72. The method of claim 68 comprising directing a beam of light pulses on the surface, the pulses characterized by a pulse length sufficient to create positive ions, ablate the surface, and heat the surface adjacent to the ablated site to form a liquid layer.

73. The method of claim 68 comprising heating the entire substrate to a temperature just below its melting point.

74. The method of claim 68 comprising forming the receptors in a body of material characterized by a characteristic filtering wavelength.

75. The method of claim 68 comprising coating a surface of the substrate with a layer of filter material.

76. The method of claim 68 comprising coating a surface of the substrate with a layer of a reflective material.

77. The method of claim 68 comprising writing at least one waveguide in the substrate.

78. The method of claim 74 comprising writing at least one waveguide in the body of the material.

79. The method of claim 68 comprising coating at least a portion of the surface with a hydrophobic or liquiphobic material prior to forming the receptor.

80. The method of claim 72 comprising coating at least a portion of the surface with a hydrophobic or liquiphobic material prior to forming the receptor.

81. The method of claim 77 comprising coating at least a portion of the surface with a hydrophobic or liquiphobic material prior to forming the receptor.

82. The method of claim 79 comprising coating at least a portion of the surface with a hydrophobic or liquiphobic material prior to forming the receptor.

83. A method of creating an array of microstructures in a material for use in molecular sequencing comprising:
directing at least one pulse of light having a pulse width less than 100 ps. from a first laser to at least one localized area to form a receptor.

84. The method of claim 83 wherein directing at least one pulse of light comprises ablating a portion of the surface to form the receptor.

85. The method of claim 83 comprising heating the surface during the step of directing a beam of less than 100 ps pulses of light to the localized area.

86. The method of claim 83 comprising melting and resolidifying at least a portion of the substrate.

87. The method of claim 83 comprising directing a beam of light pulses on the surface characterized by a pulse length sufficient to create positive ions, ablate the surface, and heat the surface adjacent to the ablated site to form a liquid layer.

88. The method of claim 83 comprising heating at least a portion of the micro assay plate to a temperature just below its melting point when machining it with light pulses.

89. The method of claim 83 comprising forming a receptor in a material characterized by a characteristic filtering wavelength.

90. The method of claim 83 comprising coating a surface with a layer of filter material.

91. The method of claim 83 comprising coating a surface with a layer of a reflective material.

92. The method of claim 83 comprising writing waveguide channels in the material.

93. The method of claim 89 comprising writing at least one waveguide in the body of the material.

94. The method of claim 83 comprising coating at least a portion of the surface with a hydrophobic or liquiphobic material prior to forming the receptor.

95. The method of claim 87 comprising coating at least a portion of the surface with a hydrophobic or liquiphobic material prior to forming the receptor.

96. The method of claim 92 comprising coating at least a portion of the surface with a hydrophobic or liquiphobic material prior to forming the receptor.

97. The method of claim 93 comprising coating at least a portion of the surface with a hydrophobic or liquiphobic material prior to forming the receptor.

98. A method of creating a receptor in a substrate for use cellular electrophysiology comprising: directing at least one pulse of light having a pulse width less than 100 ps. from a first laser to at least one localized area on the surface of the plate to form a cell receptor.

99. The method of claim 98 wherein directing at least one pulse of light comprises ablating a portion of the surface to create the cell receptor.

100. The method of claim 98 comprising heating the surface during the step of directing a beam of less than 100 ps pulses of light on the surface of the substrate.

101. The method of claim 98 comprising melting and resolidifying at least a portion of the surface of the receptor.

102. The method of claim 98 comprising directing a beam of light pulses on the surface characterized by a pulse length sufficient to create positive ions, ablate the surface, and heat the surface adjacent to the ablated site to form a liquid layer.

103. The method of claim 98 comprising heating at least a portion of the substrate to a temperature just below its melting point when directing the at least one pulse of light on the substrate.

104. The method of claim 98 in which the substrate comprises a body of glass characterized by a characteristic filtering wavelength.

105. The method of claim 98 comprising coating a surface of the substrate with a layer of filter material.

106. The method of claim 98 comprising coating a surface of the substrate with a layer of a reflective material.

107. The method of claim 98 comprising writing waveguide channels in the substrate.

108. The method of claim 98 comprising forming a receptor in a material characterized by a characteristic filtering wavelength.

109. The method of claim 98 comprising writing at least one waveguide in the body of the material.

110. The method of claim 108 comprising writing at least one waveguide in the body of the material.

111. The method of claim 98 comprising coating at least a portion of the surface with a hydrophobic or liquiphobic material prior to forming the receptor.

112. The method of claim 108 comprising coating at least a portion of the surface with a hydrophobic or liquiphobic material prior to forming the receptor.

113. The method of claim 109 comprising coating at least a portion of the surface with a hydrophobic or liquiphobic material prior to forming the receptor.

114. The substrate of claim 30 in which the coating comprises alkane thiols or polyethylene glycol.
